(19) United States
(12) Patent Application Publication
(10) Pub. No.: US 2012/0060846 A1
(43) Pub. Date: Mar. 15, 2012

(54) PILLOW

(75) Inventors: Michael S. Leoniak, Belle Meade, NJ (US); Timothy Fitzgerald, Rienzi, MS (US); Maria Sigmond, Oceanport, NJ (US)

(73) Assignee: SLEEP INNOVATIONS, INC., West Long Branch, NJ (US)

(21) Appl. No.: 13/231,595

(22) Filed: Sep. 13, 2011

Related U.S. Application Data
(60) Provisional application No. 61/382,361, filed on Sep. 13, 2010.

(51) Int. Cl. A47G 9/10; A61F 5/56

(52) U.S. Cl. 128/845; 5/636

(57) ABSTRACT

A pillow is provided that is effective in reducing and/or eliminating a user's snoring proclivity. The disclosed pillow also contributes to better sleeping outcomes, e.g., by increasing air flow into the lungs relative to conventional pillows. The pillow features a contoured upper surface that defines a head-receiving region that is bounded by raised crests of differing elevations. The pillow may be fabricated from foam materials that provide desirable levels of deformation and resilience.

### BACKGROUND

This document relates to a pillow. Individuals generally exhibit varying tendencies to snore when asleep. Individuals with a greater tendency to snore may experience less restful sleep and may impact upon the ability of others to sleep comfortably. Snoring may be caused by vibration of obstructive tissue in the airway, e.g., the soft palate and/or the uvula. Snoring may also be caused by nasal obstruction with vibration of nasal tissues and/or narrowing behind the tongue with vibration between the tongue and the back of the throat.

In addressing snoring issues, individuals have investigated behavior treatment, surgical treatment, and the use of devices aimed at snoring causes and/or symptoms. Exemplary behavioral treatments have included weight loss, avoiding alcohol and sleeping pills before sleep, and avoiding sleeping on the back (e.g., by placing an object on the individual's back such as a tennis ball). Medical interventions aimed at snoring reduction typically involve removal of relevant tissue. Such procedures include somnoplasty of the palate and uvula, laser uvulopalatoplasty (LAUP), and radiofrequency ablation.

Devices aimed at snoring prevention include jaw straps that are intended to force a user's mouth to remain shut while sleeping and devices that cause a user's oral airway to remain forcibly obstructed by acting as an airflow barrier. Unfortunately, these devices can be uncomfortable and can even inflict or promote injury, including causing jaw clenching, tension headaches, and temporomandibular joint (TMJ) disorders. Other devices include nasal passage-expanding strips, which are applied to the outside of the nose and use a strong adhesive to open the nostrils wider for quieter breathing.

Other methods of reducing snoring tendencies of individuals are disclosed in, for example, U.S. Pat. No. 4,536,905 to De Santis, which discloses a pillow that includes a semi-soft brace. The De Santis brace tends to force a user's head to one side or the other, but its overall design has several disadvantages, including a potential for causing a user to experience a sore neck.

U.S. Pat. No. 4,748,702 to Sandler discloses a pillow that features two intersecting channels formed in its upper face. One channel is intended to accommodate the user's head and the other is intended to accommodate the user's neck. A relatively hard object is located in or under the first channel and also adjacent the second channel. The relatively hard object functions so as to make it uncomfortable for a user to rest the back of the user's head on the hard object, while not being relatively uncomfortable if the user moves his/her head to one side or the other. The Sandler pillow is designed so that when the user's head is on one side or the other thereof, a part of the pillow engages the user's lower jaw, causing the user's mouth to be closed.

Cervical pillows may also be used to provide support for the head and neck of a user. Such pillows sometimes include a central channel for receiving the head of a user, raised front and rear portions, and another channel of lesser depth than the first in the raised front portion for providing support for the neck of the user.

Despite efforts to date, a need remains for effective snore-reduction techniques and/or products. In addition, a need remains for techniques and/or products that improve oxygenation and/or reduce snoring behavior while simultaneously promoting effective sleep patterns. These and other needs may be satisfied by the disclosed pillow.

### SUMMARY

The present disclosure is directed to a pillow that is effective in minimizing and/or eliminating a user's snoring proclivity. The disclosed pillow also contributes to better sleeping outcomes, e.g., by increasing air flow into the lungs relative to conventional pillow. In some embodiments, the pillow is effective in reducing and/or eliminating a user's snoring proclivity. In some embodiments, the pillow may contribute to better sleeping outcomes, e.g., by increasing air flow into the lungs relative to conventional pillows.

According to exemplary embodiments of the present disclosure, a pillow may include a first raised crest and a second raised crest that define a valley region therebetween. In some embodiments, a pillow may include a first raised crest and a second raised crest that define a valley region therebetween, wherein no inflection force has been exerted on the pillow. When a user positions his/her neck on the higher of the two crests (e.g., first raised crest) and his/her head in the valley defined therebetween, the user's neck, shoulders and chest are raised relative to the overall sleep surface, thereby relieving pressure on the soft palate and uvula, permitting the chest to expand more readily, and increasing/improving airflow in connection with the user's breathing activities. The angular orientation of a downwardly inclined surface between the crest(s) into the intermediate valley region may facilitate greater airflow to the lungs, which may translate to increased blood oxygen levels and a better night's sleep. In an embodiment of the present disclosure, the angle of a downwardly inclined surface between the neck-supporting crest into the intermediate valley region of the disclosed pillow is about 15°-20° relative to horizontal. Specific examples of the angle of the downwardly inclined surface may be about 15°, about 16°, about 17°, about 18°, about 19°, about 20°, or a range encompassed by any two of these values. In an embodiment, the transition surface of the pillow between the front face and the peak of the first crest is about 15°-20° relative to horizontal. In some embodiments, the angle of the transition surface may be about 15°, about 16°, about 17°, about 18°, about 19°, about 20°, or a range encompassed by any two of these values.

The disclosed pillow may be fabricated from a base foam material and an outer foam material. In some embodiments, the base foam material may exhibit a greater density than the outer foam material. Thus, for example, the base foam material may be a conventional polyurethane material exhibiting a density of about 2.75 to 3.3 lbs/ft³, and the outer foam material may be a viscoelastic foam material exhibiting a density of about 1.95 to about 2.05 lbs/ft³. Optionally, one of the first or second foam materials, or a third material, may be a gel-foam mixture that includes viscoelastic foam having gel particles dispersed throughout the foam. In some embodiments, the first, second, and/or third foam materials may be bonded or otherwise adhered to each other to define the final pillow product.

Further features and benefits will be apparent by reference to the drawings and ensuing detailed description of embodiments of the invention. The exclusive rights which are claimed are set forth in the numbered claims following the detailed description of the preferred embodiment.

### BRIEF DESCRIPTION OF THE DRAWINGS

To assist those of ordinary skill in the art in making and using the disclosed pillow, reference is made to the accompanying figures, wherein:

FIG. 1 provides a top view of an exemplary pillow according to the present disclosure.

FIG. 2 provides a front view of an exemplary pillow according to the present disclosure.

FIG. 3 provides a cut profile view of an exemplary pillow according to the present disclosure.

FIG. 4 provides a side view of an exemplary pillow according to the present disclosure.

FIG. 5 provides a perspective view of an exemplary pillow according to the present disclosure.

FIG. 6 is a perspective view of an exemplary pillow according to the present disclosure.

### DETAILED DESCRIPTION

The term “animal,” “patient,” “user,” or “user” as used herein includes, but is not limited to, humans and non-human vertebrates such as wild, domestic and farm animals. Preferably, the term “user,” “patient,” “user,” or “animal” refers to humans.

The terms “treating” and “to treat” as used herein may refer to both therapeutic treatment or prophylactic or preventative measures, wherein the object is to prevent or minimize (lessen) an undesired physiological condition, disorder or disease, or to obtain beneficial or desired clinical results. In some embodiments, the term may refer to both treating and preventing. For the purposes of this disclosure, beneficial or desired clinical results include, but are not limited to, prevention of symptoms; prevention of disorders associated with the condition; alleviation of symptoms; diminishment of the extent of the condition, disorder or disease; elimination of causation (temporary or permanent) of the condition, disorder or disease state; and amelioration of the condition, disorder or disease state. Treatment includes eliciting a clinically significant response without excessive levels of side effects.

As used herein, the singular forms “a,” “an,” and “the” include plural reference unless the context clearly dictates otherwise. Thus, for example, reference to a “foam material” is a reference to one or more foam materials and equivalents thereof known to those skilled in the art, and so forth.

As used herein, the terms “increase” and “decrease” mean, respectively, to cause a significant increase or decrease of at least 1%, 2%, or 5%. The terms “increase” and “decrease” mean, respectively, to cause a statistically significant (i.e., p<0.15) increase or decrease of at least 1%, 2%, or 5%

As used herein, the recitation of a numerical range for a variable is intended to convey that the invention may be practiced with the variable equal to any of the values within that range. Thus, for a variable which is inherently discrete, the variable is equal to any integer value within the numerical range, including the end-points of the range. Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, 10⁻¹², 10⁻¹¹, 10⁻¹⁰, 10⁻⁹, 10⁻⁸, 10⁻⁷, 10 ⁻⁶, 10⁻⁵, 10⁻⁴ or any other real values≧0 and ≦2 if the variable is inherently continuous.

As used herein, unless specifically indicated otherwise, the word “or” is used in the inclusive sense of “and/or” and not the exclusive sense of “either/or.”

As used herein, the term “about” means plus or minus 10% of the numerical value of the number with which it is being used. Therefore, about 50% means in the range of 45% to 55%.

As used in this document, the term “gel-foam” means a foam comprising a gel dispersed within. The term “gel” as used in this document means any liquid-extended polymer composition. In some aspects, the gel composition may contain a three-dimensional network of cross-linked molecular chains (gels), it may merely behave as it if contained such a network (gelloids). Examples of gels used in aspects of the invention include, without limitation, oil-extended triblock copolymer compositions such as that disclosed in U.S. Pat. No. 3,485,787, U.S. Pat. No. 3,676,387, U.S. Pat. No. 3,827,999, U.S. Pat. No. 4,176,240, U.S. Pat. No. 4,259,540, U.S. Pat. No. 4,351,913, U.S. Pat. No. 4,432,607, U.S. Pat. No. 4,492,428, U.S. Pat. No. 4,497,538, U.S. Pat. No. 4,509,821, U.S. Pat. No. 4,709,982, U.S. Pat. No. 4,716,183, U.S. Pat. No. 4,833,193, U.S. Pat. No. 4,942,270, U.S. Pat. No. 5,149,736, U.S. Pat. No. 5,331,036, and U.S. Pat. No. 5,994,450; and thermoplastic elastomer gelatinous compositions such as that disclosed in U.S. Pat. No. 4,369,284, U.S. Pat. No. 4,618,213, U.S. Pat. No. 5,262,468, U.S. Pat. No. 5,508,334, U.S. Pat. No. 5,153,254, U.S. Pat. No. 5,334,646, U.S. Pat. No. 5,239,273, U.S. Pat. No. 5,475,890, and U.S. Pat. No. 5,336,708. Each of the above references are hereby incorporated by reference.

Gel-foams may be made by blending a gel (which would be in particle form, optionally encapsulated with a material to preserve the integrity of the gel while it is suspended in the foam) with a polyol to form a blend. In some embodiments, the gel may be encapsulated before being blended with the polyol. Compositions used for encapsulating the gel may comprise polyol, fabric, plastic, elastomers, thermoplastic materials, foam, or the like. The gel particles may be dispersed and suspended throughout the foam composition.

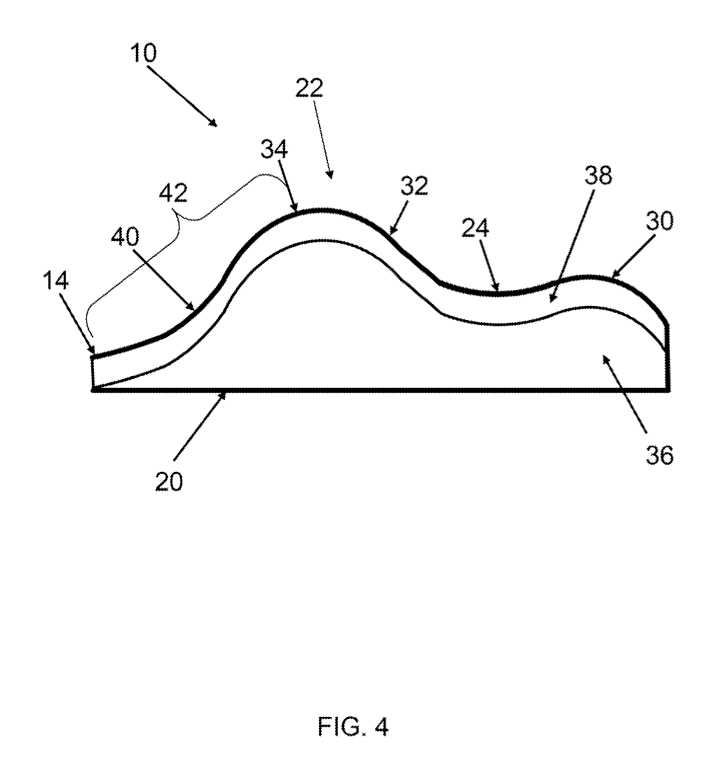

In some embodiments, the terms “point of inflection” is used to refer to the region of the front face of the pillow positioned between the pillow's highest crest and the front face of the pillow defined by the points at which the first negative slope proximate to the first crest transitions from a decreasing negative slope to an increasing negative slope. The point of inflection is proximate to the area of the pillow identified by number 40 in FIG. 4.

In some embodiments, the “transition surface” is the region of the front face of the pillow that transitions from the sloping faces of the concave region to the convex region of the first crest. In some embodiments, the transition surface 42 comprises the point of inflection 40.

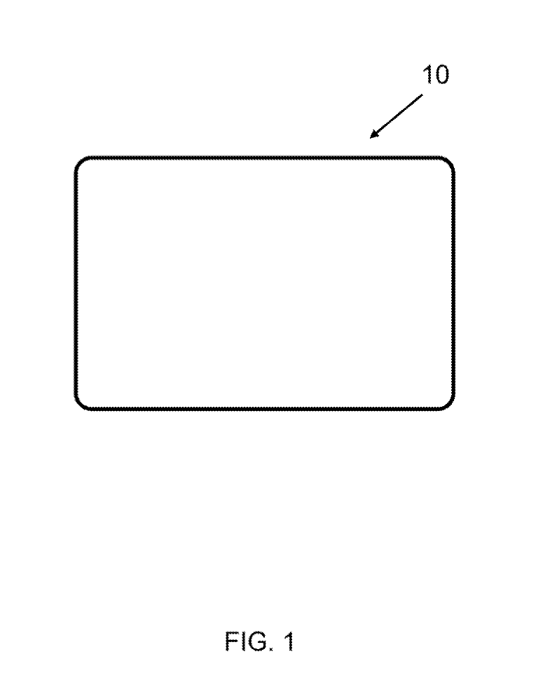

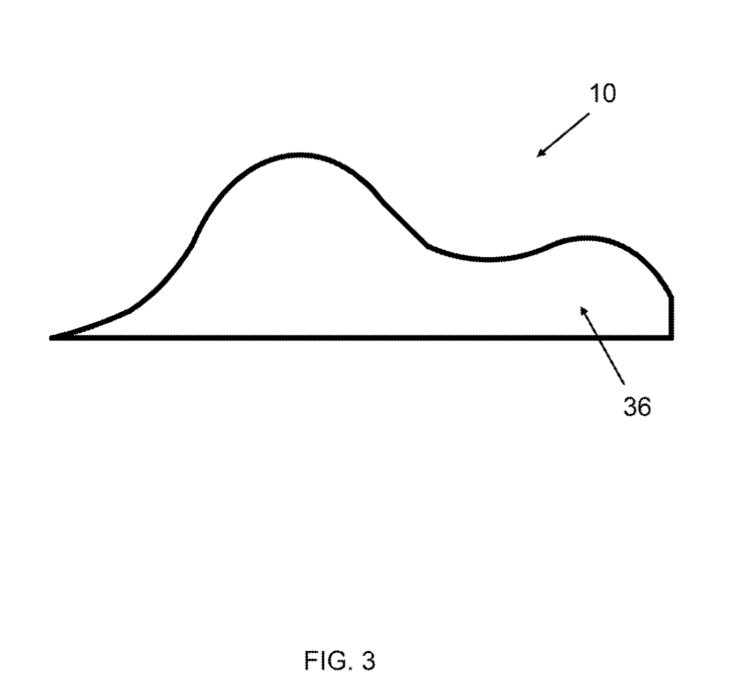

The pillow of the present disclosure is effective in minimizing and/or eliminating a user's snoring proclivity. The disclosed pillow also contributes to better sleeping outcomes, e.g., by increasing air flow into the lungs relative to conventional pillows. According to exemplary embodiments of the present disclosure, and referring to FIGS. 1-5, the pillow features a first or neck-supporting crest 34 and second crest 30 that define a valley region 24 therebetween. In some embodiments, the neck-supporting crest 34 may have an elevation that is higher than that of the second crest 30. In some embodiments, the neck-supporting crest 34 may have an elevation that is about 38% higher than that of the second crest 30.

When a user positions his/her neck over the neck-supporting crest 34 and his/her head into the valley region 24, the user's neck, shoulders and chest are raised relative to the overall sleep surface, thereby relieving pressure on the soft palate and uvula, permitting the chest to expand more readily, and increasing/improving airflow in connection with the user's breathing activities. The angular orientation of a downwardly inclined surface 32 between the crest(s) into the intermediate valley region 24 may facilitate greater airflow to the lungs, which may translate to increased blood oxygen levels and a better night's sleep. In an embodiment of the present disclosure, the angle of a downwardly inclined surface 32 between the neck-supporting crest 34 into the valley region 24 of the disclosed pillow is about 15°-20° relative to horizontal. Specific examples of the angle of the downwardly inclined surface 32 may be about 15°, about 16°, about 17°, about 18°, about 19°, about 20°, or a range encompassed by any two of these values. In an embodiment, a transition surface of the pillow between the front face 14 and the peak of the first crest 34 comprises an angle above the point of inflection 40 of about 15°-20° relative to horizontal. In some embodiments, the angle of the transition surface above the point of inflection 40 may be about 15°, about 16°, about 17°, about 18°, about 19°, about 20°, or a range encompassed by any two of these values.

In some embodiments, the disclosed pillow may be fabricated from at least a first foam layer and a second foam layer. In some embodiments, the first foam layer may comprise a core foam layer 36. In some embodiments, the second foam layer may comprise an outer foam layer 38. In some embodiments, the core foam layer 36 may exhibit a greater density than the outer foam layer 38. For example, the core foam layer 36 may comprise a conventional polyurethane foam exhibiting a density of about 2.75 to 3.3 lbs/ft³ and the outer foam layer 38 may comprise a viscoelastic foam exhibiting a density of about 1.95 to about 2.05 lbs/ft³. The first and second foam layers may be bonded or otherwise adhered to each other to define the final pillow product.

FIGS. 1-5 illustrate an exemplary pillow 10 that provides improved oxygenation according to the present disclosure. The pillow 10 includes a body composed of a core foam layer 36 and an outer foam layer 38. In some embodiments, the core foam layer 36 may be a material identified by Advanced Urethane Technologies (Coldwater, Miss.) as Product 2033w and the outer foam layer 38 may be a material identified by Advanced Urethane Technologies (Coldwater, Miss.) as Product KC30010NAE. Other materials may be possible. In particular embodiments, the core foam layer 36 may comprise a high resiliency foam that provides support and the outer foam layer 38 may comprise memory foam. Memory foam generally may be made of polyurethane with additional chemicals increasing its viscosity and density. Higher-density memory foam reacts to body heat, allowing it to mold to a warm human body in a few minutes. A lower-density memory foam is pressure-sensitive and molds quickly to the shape of the body

In some embodiments, the core foam layer 36 of the pillow is fabricated from a polyurethane foam. In some embodiments, the outer foam layer 38 is fabricated from a polyurethane foam. In some embodiments, the core foam layer 36 of the pillow is fabricated from a viscoelastic foam. In some embodiments, the outer foam layer 38 is fabricated from a viscoelastic foam. In some embodiments, the core foam layer 36 and outer foam layer 38 are formed from polyurethane. In some embodiments, the core foam layer 36 and outer foam layer 38 are formed from a viscoelastic material. In some embodiments, the core foam layer 36 comprises gel foam. In some embodiments, the outer foam layer 38 comprises gel foam. In some embodiments, the core foam layer 36 and the outer foam layer 38 comprise gel foam.

According to exemplary embodiments of the present disclosure, the core foam layer 36 may comprise the majority of pillow 10, with an outer foam layer 38 of viscoelastic foam. In some embodiments, the outer foam layer 38 may be about 0.5 to about 1.0 inches in thickness, although alternative thicknesses may be employed such as, without limitation, about 0.6, about 0.7, about 0.75, about 0.8, or about 0.9 inches. It is further contemplated that the thickness of outer foam layer 38 may vary at different locations of anti-snore pillow 10. In some embodiments, the outer foam layer 38 may be thicker in the valley region as compared to the crests. The outer foam layer 38 may be on top of the pillow, as shown in FIG. 4. In some embodiments, the outer foam layer 38 may cover a portion or all of upper face 22. In some embodiments, the outer foam layer 38 may cover any or all of the sides (16, 18, 14, or 12) and/or base 20 of the pillow.

As shown in FIGS. 1-5, an anti-snore pillow 10 comprises a rear face 12, a front face 14, two side faces 16 and 18, a base 20 and an upper face 22. In some embodiments, base 20 may be substantially flat in design, thereby facilitating engagement with a sleep surface, e.g., a mattress. Rear face 12 may be vertical in orientation relative to base 20, but the disclosed pillow is not limited by or to such substantially vertical orientation. Side faces 16 and 18 may be vertical in orientation relative to base 20, although as with rear face 12, the present disclosure is not limited by or to such substantially vertical orientation.

Upper face 22 of anti-snore pillow 10 may include a first crest 34 and a second crest 30. A valley region 24 may be found between second crest 30 and first crest 34. In some embodiments, the valley region 24 is dimensioned to accommodate a user's head when the user's neck is positioned in substantial engagement with higher of the two crests 34. In some embodiments, the downwardly inclined surface 32 that extends from first crest 34 into valley region 24 is generally angularly oriented relative to base 20, such that pressure is relieved from the palate and uvula of the user. The angle of downwardly inclined surface 32 may be about 5° to about 20° relative to the horizontal, about 15° to about 20° relative to horizontal, and preferably about 18° relative to horizontal (i.e., base 20). In an embodiment of the present disclosure, the angle defined from the neck-supporting crest into the intermediate valley region of the disclosed pillow is about 15°-20° relative to horizontal. Specific examples of the angle defined from the neck-supporting crest to the intermediate valley region of the disclosed pillow may be about 15°, about 16°, about 17°, about 18°, about 19°, about 20°, or a range encompassed by any two of these values. In an embodiment, the surface of the pillow supporting the neck of a user is about 15°-20° relative to horizontal. In some embodiments, the angle of the surface of the pillow that supports the neck of the user may be about 15°, about 16°, about 17°, about 18°, about 19°, about 20°, or a range encompassed by any two of these values.

In the exemplary embodiment of FIGS. 1-5, valley region 24 extends from side-to-side of anti-snore pillow 10, i.e., from side face 16 to side face 18. However, alternative embodiments are contemplated wherein side walls may be provided that bound, in whole or in part, valley region 24. From the side view of FIG. 4, it is apparent that valley region 24 defines a substantially concave geometry such that sloping faces are defined to transition to crests 30, 34. In some embodiments, second crest 30 may be of equal (or substantially equal) height. In some embodiments, first crest 34 may be of greater height relative to base 20 as compared to second crest 30. In some embodiments, the first crest 34 may be about 30% to about 45% higher than the second crest 30. In some embodiments, the first crest 34 may be about 35% to about 40% higher than the second crest 30. In some embodiments, the first crest 34 may be about 38% higher than the second crest 30.

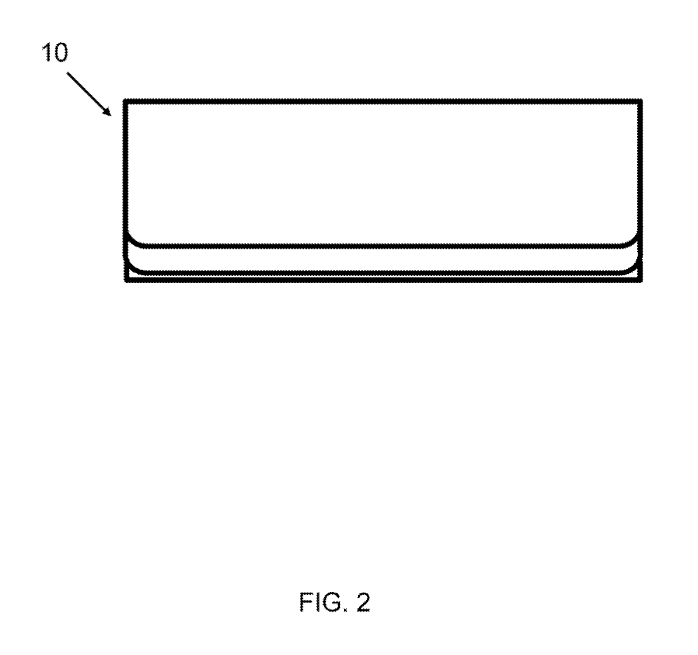

In some embodiments of the present disclosure, first crest 34 has a height of about 118.32 millimeters (mm) and second crest 30 has a height of about 85.5 mm. Other sizes are possible. For example, either or both of the crests may have heights ranging from about 3.5 to about 4 inches, about 4 to about 5 inches, about 3.5 to about 5 inches, about 3.5 to about 4 inches or other sizes. The overall width of the disclosed anti-snore pillow may vary widely. As shown in FIG. 2, in an exemplary embodiment, the pillow 10 may be about 487.3 mm (about 20 inches) wide from side face 16 to side face 18. The depth of the snore pillow 10 may also vary from implementation to implementation. In an exemplary embodiment, the distance from front face 14 to rear face 12 may be about 362.69 mm (about 15 inches) as shown in FIG. 1.

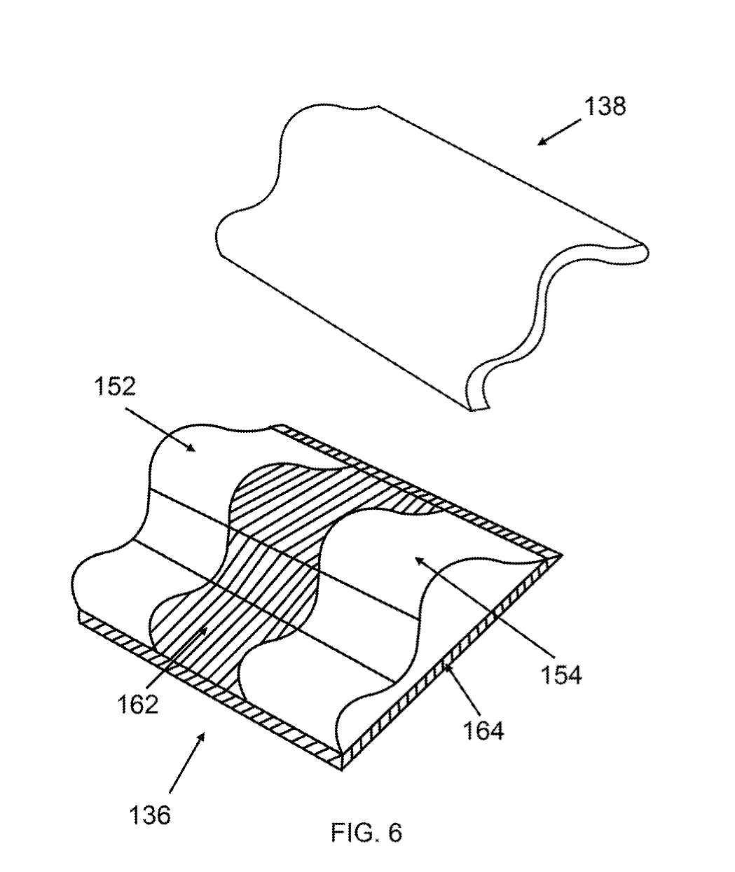

FIG. 6 describes an embodiment in which a combination of materials may be used for the base layer. As shown in FIG. 6, in some embodiments, a core foam layer 136 may include a central section 162 made of a first foam material and two side sections 152, 154 made of a second foam material. The base 164 also may be made of the second foam material. An outer foam layer 138 may be made of the second foam material or of a third foam material. Any of the first, second or third foam materials may be consistent with any of the foams described above in this disclosure, including, but not limited to, base foam, conventional polyurethane foam, viscoelastic foam, and/or gel-foam.

As shown in FIG. 6, exemplary dimensions of the various sections may include an overall depth (including base) of about 342.63 mm (about 13.5 inches). In some embodiments, the depth of the side sections 152 and 154, and central section 162 may be about 281.64 mm (about 11 inches). In some embodiments, the overall depth of the pillow may be from about 10 to about 15 inches, from about 5 to about 15 inches, from about 10 to about 17 inches, from about 5 to about 17 inches, about 10 inches, about 13 inches, about 15 inches, about 17 inches, or a range between any two of these values. In some embodiments, the depth of a pillow section may be from about 10 to about 15 inches, from about 5 to about 15 inches, from about 10 to about 17 inches, from about 5 to about 17 inches, about 10 inches, about 11 inches, about 13 inches, about 15 inches, about 17 inches, or a range between any two of these values. In some embodiments, the central section 162 may have a width that is about equal to that of the combined width of the side sections 152 and 154. For example, in the embodiment shown the central section 162 has a width of 254 mm (about 10 inches), while each side section (152, 154) has a width of 133.35 mm (about 5.25 inches). In some embodiments, each of the sections may be of equal width. In some embodiments, at least two of the sections may be of unequal width. In some embodiments, the width of a section may be from about 1 inch to about 15 inches, from about 5 to about 10 inches, from about 3 to about 15 inches, from about 5 to about 15 inches, from about 1 to about 10 inches, from about 3 to about 10 inches, or the like. Other sizes are possible.

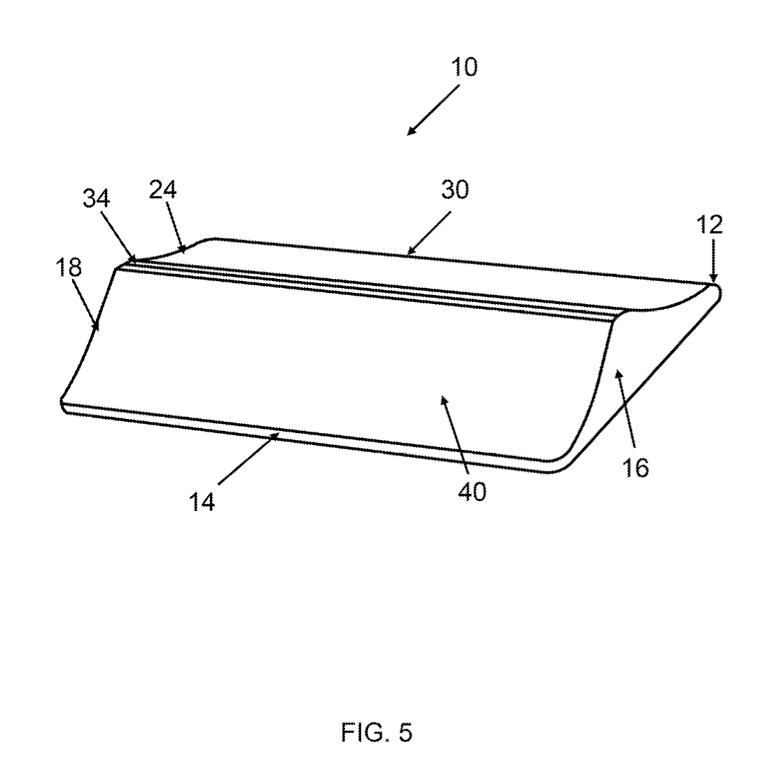

In some embodiments, an anti-snore pillow comprises a pillow body comprising a first crest, a second crest, a valley region positioned between the first and second crests. In some embodiments, an anti-snore pillow comprises a pillow body comprising a first crest, a second crest, a valley region positioned between the first and second crests, wherein no inflection force has been exerted on the pillow. In some embodiments, the pillow body retains shape when no inflection force has been exerted. As used herein, “inflection force” refers to exposing a material to load. For example, “inflection force” may include placing the head or other body part of a user onto the pillow, placing any weighted item onto the pillow, or the like. As used herein, the term “inflection force density” refers to a measure of the material's performance under load (inflection force). In some embodiments, the pillow body may further comprise a base, a transition surface positioned between the top of the first crest to the base and opposite to the valley region, and a front face positioned between the front face and the transition surface. In some embodiments, the pillow body comprises a core foam layer and an outer foam layer. In some embodiments, the first crest and the valley region cooperate to reduce snoring behavior of a user when a neck of the user is positioned in substantial contact with the first crest and a head of the user is positioned in substantial contact with the valley region. In some embodiments, the neck of the user is positioned in substantial contact with the first crest at an angle of between about 10° to about 20° relative to the horizontal of the base of the pillow. In some embodiments, the neck of the user is positioned in substantial contact with the first crest at an angle of between about 11° to about 20° relative to the horizontal of the base of the pillow. In some embodiments, the neck of the user is positioned in substantial contact with the first crest at an angle of between about 12° to about 20° relative to the horizontal of the base of the pillow. In some embodiments, the reduction of snoring of a user is caused when the neck of the user is positioned in substantial contact with the first crest at an angle of between about 13° to about 20° relative to the horizontal of the base of the pillow. In some embodiments, the reduction of snoring of a user is caused when the neck of the user is positioned in substantial contact with the first crest at an angle of between about 14° to about 20° relative to the horizontal of the base of the pillow. In some embodiments, the reduction of snoring of a user is caused when the neck of the user is positioned in substantial contact with the first crest at an angle of between about 15° to about 20° relative to the horizontal of the base of the pillow. In some embodiments, the reduction of snoring of a user is caused when the neck of the user is positioned in substantial contact with the first crest at an angle of between about 16° to about 20° relative to the horizontal of the base of the pillow. In some embodiments, the reduction of snoring of a user is caused when the neck of the user is positioned in substantial contact with the first crest at an angle of between about 17° to about 20° relative to the horizontal of the base of the pillow. In some embodiments, the reduction of snoring of a user is caused when the neck of the user is positioned in substantial contact with the first crest at an angle of between about 18° to about 20° relative to the horizontal of the base of the pillow. In some embodiments, the reduction of snoring of a user is caused when the neck of the user is positioned in substantial contact with the first crest at an angle of about 15° relative to the horizontal of the base of the pillow. In some embodiments, the reduction of snoring of a user is caused when the neck of the user is positioned in substantial contact with the first crest at an angle of about 16° relative to the horizontal of the base of the pillow. In some embodiments, the reduction of snoring of a user is caused when the neck of the user is positioned in substantial contact with the first crest at an angle of about 17° relative to the horizontal of the base of the pillow. In some embodiments, the reduction of snoring of a user is caused when the neck of the user is positioned in substantial contact with the first crest at an angle of about 18° relative to the horizontal of the base of the pillow. In some embodiments, the reduction of snoring of a user is caused when the neck of the user is positioned in substantial contact with the first crest at an angle of about 19° relative to the horizontal of the base of the pillow. In some embodiments, the neck of the user may be positioned in substantial contact with the first crest at an angle of about 20° relative to the horizontal of the base of the pillow. In some embodiments, the above-mentioned angle is measured from a point of inflection on the front face (proximate to line number 40 in FIG. 5) to the front face (proximate to line number 14 in FIG. 5) and relative to the base. In some embodiments, the point of inflection relevant to calculate the angle measurement is on the front face of the pillow.

As used herein, the term “inflection force density” refers to a measure of the material's performance when exposed to an inflection force (i.e. under load). Further embodiments of the invention include a pillow with the core foam layer and the outer foam layer that have certain properties. In some embodiments, the pillow comprises a core foam layer comprising the product identified as 2033w according to records on file with Advanced Urethane Technologies (Coldwater, Miss.). In some embodiments, the pillow comprises an outer foam layer comprising the product identified as KC30010NAE according to records on file with Advanced Urethane Technologies (Coldwater, Miss.). In some embodiments, the pillow may comprise a core foam layer that has an inflection force density between about 10 to about 50 at a 25% deflection. In some embodiments, the pillow may comprise a core foam layer that has an inflection force density between about 10 to about 50 at a 25% deflection. In some embodiments, the pillow comprises a core foam layer that has an inflection force density between about 30 to about 40 at a 25% deflection. In some embodiments, the pillow comprises a core foam layer that has an inflection force density between about 32 to about 38 at a 25% deflection. In some embodiments, the pillow comprises a core foam layer that has an inflection force density of about 31 at a 25% deflection. In some embodiments, the pillow comprises a core foam layer that has an inflection force density of about 32 at a 25% deflection. In some embodiments, the pillow comprises a core foam layer that has an inflection force density of about 33 at a 25% deflection. In some embodiments, the pillow comprises a core foam layer that has an inflection force density of about 34 at a 25% deflection. In some embodiments, the pillow comprises a core foam layer that has an inflection force density of about 35 at a 25% deflection. In some embodiments, the pillow comprises a core foam layer that has an inflection force density of about 36 at a 25% deflection. In some embodiments, the pillow comprises a core foam layer that has an inflection force density of about 37 at a 25% deflection. In some embodiments, the pillow comprises a core foam layer that has an inflection force density of about 38 at a 25% deflection. In some embodiments, the pillow comprises a core foam layer that has an inflection force density of about 39 at a 25% deflection. In some embodiments, the pillow comprises a core foam layer that has an inflection force density of about 40 at a 25% deflection.

In some embodiments, the density of the core foam layer is between about 0.10 to about 10.00 pounds per cubic foot. In some embodiments, the density of the core foam layer is between about 1.00 to about 3.00 pounds per cubic foot. In some embodiments, the density of the core foam layer is between about 1.50 to about 2.10 pounds per cubic foot. In some embodiments, the density of the core foam layer is about 1.10 pounds per cubic foot. In some embodiments, the density of the core foam layer is about 1.20 pounds per cubic foot. In some embodiments, the density of the core foam layer is about 1.30 pounds per cubic foot. In some embodiments, the density of the core foam layer is about 1.40 pounds per cubic foot. In some embodiments, the density of the core foam layer is about 1.50 pounds per cubic foot. In some embodiments, the density of the core foam layer is about 1.60 pounds per cubic foot. In some embodiments, the density of the core foam layer is about 1.70 pounds per cubic foot. In some embodiments, the density of the core foam layer is about 1.80 pounds per cubic foot. In some embodiments, the density of the core foam layer is about 1.90 pounds per cubic foot. In some embodiments, the density of the core foam layer is about 2.00 pounds per cubic foot. In some embodiments, the density of the core foam layer is about 2.10 pounds per cubic foot.

Further embodiments of the invention include a pillow with a base layer, wherein the minimum percent resilience of the core foam layer is at least about 30%. In some embodiments, the base layer has a minimum percent resilience of at least about 40%. In some embodiments, the base layer has a minimum percent resilience of at least about 50%. In some embodiments, the minimum percent resilience of the core foam layer is between about 35% and about 50%. In some embodiments, the minimum percent resilience of the core foam layer is between about 40% and about 50%. In some embodiments, the minimum percent resilience of the core foam layer is between about 40% and about 45%.

In some embodiments, the outer foam layer may have an inflection force density between about 1 to about 20 at a 25% deflection. In some embodiments, the outer foam layer has an inflection force density between about 5 to about 20 at a 25% deflection. In some embodiments, the outer foam layer has an inflection force density between about 5 to about 15 at a 25% deflection. In some embodiments, the outer foam layer has an inflection force density between about 5 to about 13 at a 25% deflection. In some embodiments, the outer foam layer has an inflection force density between about 6 to about 13 at a 25% deflection. In some embodiments, the outer foam layer has an inflection force density between about 7 to about 13 at a 25% deflection. In some embodiments, the outer foam layer has an inflection force density between about 8 to about 13 at a 25% deflection. In some embodiments, the outer foam layer has an inflection force density between about 8.5 to about 12.5 at a 25% deflection.

Further embodiments of the invention include a pillow with an outer foam layer that may be from about 0.10 to about 10.00 pounds per cubic foot. In some embodiments, the density of the outer foam layer is between about 1.00 to about 5.00 pounds per cubic foot.

In some embodiments, the density of the outer foam layer is between about 2.50 to about 3.50 pounds per cubic foot. In some embodiments, the density of the outer foam layer is between about 1.00 to about 5.00 pounds per cubic foot. In some embodiments, the density of the outer foam layer is about 2.50 pounds per cubic foot. In some embodiments, the density of the outer foam layer is about 2.55 pounds per cubic foot. In some embodiments, the density of the outer foam layer is about 2.60 pounds per cubic foot. In some embodiments, the density of the outer foam layer is about 2.65 pounds per cubic foot. In some embodiments, the density of the outer foam layer is about 2.70 pounds per cubic foot. In some embodiments, the density of the outer foam layer is about 2.75 pounds per cubic foot. In some embodiments, the density of the outer foam layer is about 2.80 pounds per cubic foot. In some embodiments, the density of the outer foam layer is about 2.85 pounds per cubic foot. In some embodiments, the density of the outer foam layer is about 2.90 pounds per cubic foot. In some embodiments, the density of the outer foam layer is about 2.95 pounds per cubic foot. In some embodiments, the density of the outer foam layer is about 3.00 pounds per cubic foot. In some embodiments, the density of the outer foam layer is about 3.05 pounds per cubic foot. In some embodiments, the density of the outer foam layer is about 3.10 pounds per cubic foot. In some embodiments, the density of the outer foam layer is about 3.15 pounds per cubic foot. In some embodiments, the density of the outer foam layer is about 3.20 pounds per cubic foot. In some embodiments, the density of the outer foam layer is about 3.25 pounds per cubic foot. In some embodiments, the density of the outer foam layer is about 3.30 pounds per cubic foot. In some embodiments, the density of the outer foam layer is about 3.35 pounds per cubic foot. In some embodiments, the density of the outer foam layer is about 3.40 pounds per cubic foot.

In some embodiments the pillow comprises an outer foam layer wherein a minimum percent resilience of the outer foam layer is at least about 0%. In some embodiments the pillow comprises an outer foam layer wherein a minimum percent resilience of the outer foam layer is at least about 0%. In some embodiments the pillow comprises an outer foam layer wherein a minimum percent resilience of the outer foam layer is at least about 1%. In some embodiments the pillow comprises an outer foam layer wherein a minimum percent resilience of the outer foam layer is at least about 2%. In some embodiments the pillow comprises an outer foam layer wherein a minimum percent resilience of the outer foam layer is at least about 3%. In some embodiments the pillow comprises an outer foam layer wherein a minimum percent resilience of the outer foam layer is at least about 4%. In some embodiments the pillow comprises an outer foam layer wherein a minimum percent resilience of the outer foam layer is at least about 5%. In some embodiments the pillow comprises an outer foam layer wherein a minimum percent resilience of the outer foam layer is at least about 6%. In some embodiments the pillow comprises an outer foam layer wherein a minimum percent resilience of the outer foam layer is at least about 7%. In some embodiments the pillow comprises an outer foam layer wherein a minimum percent resilience of the outer foam layer is at least about 8%. In some embodiments the pillow comprises an outer foam layer wherein a minimum percent resilience of the outer foam layer is at least about 9%. In some embodiments the pillow comprises an outer foam layer wherein a minimum percent resilience of the outer foam layer is at least about 10%. In some embodiments, the pillow comprises an outer foam layer wherein a minimum percent resilience of the outer foam layer is between about 0% to about 10%. In some embodiments, the pillow comprises an outer foam layer wherein a minimum percent resilience of the outer foam layer is between about 0% to about 10%. In some embodiments, the pillow comprises an outer foam layer wherein a minimum percent resilience of the outer foam layer is between about 0% to about 5%. In some embodiments, the pillow comprises an outer foam layer wherein a minimum percent resilience of the outer foam layer is between about 0% to about 2%.

In some embodiments, the combination of the neck of the user positioned in substantial contact with the first crest and the angle measured from the front face to the point of inflection causes increased air flow to the user's lungs. In some embodiments, the combination of the neck of the user positioned in substantial contact with the first crest and the angle measured from the front face to the point of inflection causes increased air flow to the user's respiratory system. In some embodiments, the combination of the neck of the user positioned in substantial contact with the first crest and the angle measured from the front face to the point of inflection causes reduction of the vibration frequency or intensity of obstructive tissue in the airway of the user. In some embodiments, the combination of the neck of the user positioned in substantial contact with the first crest and the angle measured from the front face to the point of inflection causes relief of pressure on the chest and neck of the user. In some embodiments, the combination of the neck of the user positioned in substantial contact with the first crest and the angle measured from the front face to the point of inflection causes increased oxygen saturation in the user. In some embodiments, the combination of the neck of the user positioned in substantial contact with the first crest and the angle measured from the front face to the point of inflection causes decreased occurrence of snoring of the user. In some embodiments, the combination of the neck of the user positioned in substantial contact with the first crest and the angle measured from the front face to the point of inflection causes an increased duration of sleep. In some embodiments, the combination of the neck of the user positioned in substantial contact with the first crest and the angle measured from the front face to the point of inflection causes an increased duration of rapid eye movement sleep. In some embodiments, the combination of the neck of the user positioned in substantial contact with the first crest and the angle measured from the front face to the point of inflection causes a reduction of snoring and/or increased duration of sleep. In some embodiments, a method of treating snoring comprises the step of: providing a pillow that includes a pillow body comprising a first crest, a second crest, a valley region positioned between the first and second crests. In some embodiments, the pillow body may further comprise a base 16, a front face 14, a transition surface positioned between the top of the first crest 34 to the front face 14. In some embodiments, the pillow body comprises a core foam layer and an outer foam layer. In some embodiments, the method of treating snoring further comprises the step of: positioning a neck of a user in substantial contact with the first crest and a head of the user in substantial contact with the valley region. In some embodiments, a method of treating snoring comprise the steps of: providing a pillow that includes a pillow body comprising a first crest, a second crest, and a valley region positioned between the first and second crests; and positioning a neck of a user in substantial contact with the first crest and a head of the user in substantial contact with the valley region. In some embodiments, a method of treating snoring comprise the steps of: providing a pillow that includes a pillow body comprising a first crest, and a valley region positioned between the first and second crests; and positioning a neck of a user in substantial contact with the first crest and a head of the user in substantial contact with the valley region. In some embodiments, the pillow body may further comprise a second crest. In some embodiments, the pillow body may further comprise a bottom face, a transition surface positioned between the top of the first crest to the bottom face and opposite to the valley region, and a front face positioned between the bottom face and the transition surface. In some embodiments, the pillow body comprises a core foam layer and an outer foam layer. In some embodiments, the method may involve one, two, or more users. In some embodiments, a single user provides and positions himself or herself on the pillow. In some embodiments, the user may be susceptible to or diagnosed with snoring. In some embodiments, the snoring may be caused by the vibration of obstructive tissue in the airway of the user.

In some embodiments, a pillow of embodiments herein may be used in methods of treating sleep disorders including, without limitation, insomnia and sleep apnea. The invention further relates to a method of treatment and/or prevention of low oxygenation of the blood. In some embodiments, a method of treating a sleep disorder comprises the step of: providing a pillow that includes a pillow body comprising a first crest, a second crest, a valley region positioned between the first and second crests. In some embodiments, the pillow body may further comprise a bottom face, a transition surface positioned between the top of the first crest to the bottom face and opposite to the valley region, and a front face positioned between the bottom face and the transition surface. In some embodiments, the pillow body comprises a core foam layer and an outer foam layer. In some embodiments, the method of treating a sleep disorder further comprises the step of: positioning a neck of a user in substantial contact with the first crest and a head of the user in substantial contact with the valley region. In some embodiments, a method of treating a sleep disorder comprises the steps of: providing a pillow that includes a pillow body comprising a first crest, and a valley region positioned between the first and second crests; and positioning a neck of a user in substantial contact with the first crest and a head of the user in substantial contact with the valley region. In some embodiments, the pillow body may further comprise a second crest. In some embodiments, the pillow body may further comprise a bottom face, a transition surface positioned between the top of the first crest to the bottom face and opposite to the valley region, and a front face positioned between the bottom face and the transition surface. In some embodiments, the pillow body comprises a core foam layer and an outer foam layer. In some embodiments, the user may be susceptible to or diagnosed with a sleep disorder. In some embodiments, the user may be susceptible to or diagnosed with a low oxygenation of the blood. In some embodiments, the user may be susceptible to or diagnosed with sleep apnea or insomnia. In some embodiments, the user may be susceptible to or diagnosed with a sleep condition that causes light sleep, intermittent sleep, or reduced periods of rapid eye movement (REM) sleep. In some embodiments, the sleep disorder may be selected from sleep apnea, insomnia, low oxygenation of blood, reduced REM sleep, light sleep, intermittent sleep or a combination thereof.

Although the present disclosure has been described with reference to exemplary embodiments of the advantageous anti-snore pillow, the present disclosure is not limited by or to such exemplary embodiments. Rather, the present disclosure expressly encompasses modifications, alterations and/or enhancements to the disclosed embodiments, as will be readily apparent to persons of ordinary skill in the art based on the present disclosure.

1. A pillow comprising:
a pillow body including
a base;
a first crest that extends upward from the base to receive a neck of a user;
a second crest that extends upward from the base;
a valley region positioned between the first and second crests to receive a head of the user;
a front face; and
a transition surface positioned between a peak of the first crest and the front face,
wherein the transition surface comprises a point of inflection positioned on a side of the first crest proximate to the front face, and
wherein the first crest has a height greater than a height of the second crest.

2. The pillow of claim 1, wherein the pillow retains a shape of the pillow body when no inflection force is exerted.

3. The pillow of claim 1, wherein the pillow comprises a core foam layer and an outer foam layer.

4. The pillow of claim 3, wherein the core foam layer and the outer foam layer each comprise a viscoelastic foam material.

5. The pillow of claim 3, wherein the core foam layer comprises a polyurethane foam.

6. The pillow of claim 3, wherein the core foam layer has a central section fabricated from a first type of foam and side sections fabricated from a second type of foam.

7. The pillow of claim 3, wherein the core foam layer has an inflection force density between about 10 to about 50 at a 25% deflection.

8. The pillow of claim 3, wherein the core foam layer has an inflection force density between about 30 to about 40 at a 25% deflection.

9. The pillow of claim 3, wherein the outer foam layer has an inflection force density between about 5 to about 20 at a 25% deflection.

10. The pillow of claim 3, wherein the outer foam layer has an inflection force density between about 8.5 to about 12.5 at a 25% deflection.

11. The pillow of claim 1, wherein the transition surface comprises an angle above the point of inflection from about 5° to about 20° relative to the base.

12. The pillow of claim 11, wherein the angle above the point of inflection is from about 15° to about 20° relative to the base.

13. The pillow of claim 1, wherein the height of the first crest is about 30% to about 45% higher than the height of the second crest.

14. The pillow of claim 1, wherein the height of the first crest is about 38% higher than the height of the second crest.

15. The pillow of claim 1, further comprising a downwardly inclined surface between a peak of the first crest and the valley region.

16. The pillow of claim 16, wherein the downwardly inclined surface has an angle from about 15° to about 20° relative to the base.

17. A method of treating snoring, the method comprising:
providing a pillow that includes a pillow body comprising
a base;
a first crest that extends upwards from the base to receive a neck of a user;
a second crest that extends upwards from the base;
a valley region positioned between the first and second crests to receive a head of the user;
a front face; and
a transition surface positioned between a peak of the first crest and the front face,
wherein the transition surface comprises a point of inflection positioned on a side of the first crest proximate to the front face, and
wherein the first crest has a height greater than a height of the second crest; and
positioning a neck of a user in substantial contact with the first crest and a head of the user in substantial contact with the valley region.

18. A method of treating a sleep disorder, the method comprising:
providing a pillow that includes a pillow body comprising
a base;
a first crest that extends upwards from the base to receive a neck of a user;
a second crest that extends upwards from the base;
a valley region positioned between the first and second crests to receive a head of the user;
a front face; and
a transition surface positioned between a peak of the first crest and the front face,
wherein the transition surface comprises a point of inflection positioned on a side of the first crest proximate to the front face, and
wherein the first crest has a height greater than a height of the second crest; and
positioning a neck of a user in substantial contact with the first crest and a head of the user in substantial contact with the valley region.

19. The method of claim 18, wherein the sleep disorder is selected from sleep apnea, insomnia, low oxygenation of blood, reduced REM sleep, light sleep, intermittent sleep or a combination thereof.
